(19) United States
(12) Patent Application Publication
(10) Pub. No.: US 2023/0108277 A1
(43) Pub. Date: Apr. 6, 2023

(54) Intubation System and Method of Use

(71) Applicant: Kiran B. Khanolkar, Keokuk, IA (US)

(72) Inventor: Kiran B. Khanolkar, Keokuk, IA (US)

(21) Appl. No.: 17/449,688

(22) Filed: Oct. 1, 2021

(51) Int. Cl. A61M 16/04; A61B 1/00; A61B 1/273

(52) U.S. Cl. CPC A61M 16/0488; A61M 16/0463; A61M 16/0402; A61B 1/00096; A61B 1/00082; A61B 1/273

(57) ABSTRACT

A surgical system includes an endotracheal system and intubation system. The intubation system includes an introducer rod and intubation instrument. The intubation instrument includes: an esophageal tube, intubation tube, endoscopic camera tube, and balloon located distally beyond a distal end of the intubation tube. The introducer rod is insertable through the intubation tube to a position where an endoscopic camera in the endoscopic camera tube has a line of sight to a distal end of the introducer rod. The balloon is movable from a deflated condition to an inflated condition in which the balloon fully occludes the esophageal opening to block advancement of the introducer rod into the esophagus and urge the introducer rod into the trachea. The intubation tube is removable from the patient leaving behind the introducer rod, such that the introducer rod is guides an endotracheal tube of the endotracheal system into the patient's trachea.

### FIELD OF THE DISCLOSURE

The present disclosure relates to an intubation system and method of use, and more particularly, to an intubation system and method of use that is effective for difficult intubation procedures.

### BACKGROUND OF THE DISCLOSURE

Intubation is a common medical procedure performed multiple times a day in hospitals worldwide. A purpose of this procedure to ensure that an individual receives adequate oxygen necessary for survival. Several intubation procedures are deemed difficult intubations, which usually result from patients with multiple trauma, obesity, and/or excessive secretions or blood in the oropharynx.

Conventional intubation devices lack components and/or lack the most effective arrangement and orientation of certain components, which could improve the easy and efficacy of intubation procedures if present and arranged properly. Therefore, what is needed is a system and method for improved intubation especially for difficult intubation procedures.

### SUMMARY

In an illustrative embodiment, an intubation system comprises: an introducer rod and an intubation instrument, each configured to move relative to one another; wherein the intubation instrument includes: an esophageal tube that is curved and has a distal opening and a proximal opening; an intubation tube extending adjacent a portion of the esophageal tube and having a distal opening and a proximal opening; an endoscopic camera tube extending adjacent the intubation tube and including a proximal opening and a distal opening adjacent the distal opening of the intubation tube; a balloon fixed relative to the esophageal tube and extending around a circumference of the esophageal tube, wherein the balloon is distal of the distal opening of the intubation tube; wherein the introducer rod is flexible and insertable through the intubation tube; wherein the balloon is movable between: (a) an inflated condition, in which the balloon fully occludes the esophageal opening of a patient and (b) a deflated condition in which the balloon does not fully occlude the esophageal opening of the patient when positioned in the esophageal opening of the patient.

In some embodiments, the balloon is arranged relative to the intubation tube such that the introducer rod is configured to contact the balloon during movement of the introduce rod relative to the intubation instrument when the balloon is positioned in the esophagus of the patient. In some embodiments, the intubation instrument includes only a single balloon.

In some embodiments, the intubation system further comprises an endoscopic camera positioned at the distal opening of the endoscopic camera tube in an arrangement such that the endoscopic camera has a line of sight to the vocal cords of the patient.

In some embodiments, when the introducer rod is positioned in the patient's trachea and a distal end of the introducer rod is positioned distal of the distal opening of the intubation tube, the endoscopic camera has a line of sight to the distal end of the introducer rod when the balloon is in the deflated condition and in the inflated condition.

In some embodiments, the intubation system further comprises a pair of airway tubes positioned on opposite sides of the intubation tube; and each airway tube is configured to facilitate suction in the proximal direction and urge airflow in the distal direction.

In some embodiments, each airway tube includes a proximal opening, a distal opening, and a plurality of apertures defined proximal of the distal opening.

In some embodiments, the intubation system further comprises a cover surrounding each of the esophageal tube, the intubation tube, the endoscopic camera tube, a balloon insufflation tube coupled to the balloon, and the pair of airway tubes.

In some embodiments, the cover comprises a silicone material.

In some embodiments, the balloon is positioned between the distal opening of the esophageal tube and the distal opening of the intubation tube.

In another illustrative embodiment, a surgical system comprises the intubation system of described above and an endotracheal system, the endotracheal system comprising: an endotracheal tube configured to be advanced along the introducer rod when the introducer rod is positioned in the patient's trachea; and the endotracheal tube is sized and shaped such that it cannot pass through the intubation tube or the esophageal tube of the intubation instrument to be inserted in the trachea of the patient.

In another illustrative embodiment, a method of performing an procedure on a patient comprises inserting an intubation instrument through the mouth of the patient, the intubation instrument including: (i) an esophageal tube that is curved, (ii) an intubation tube extending along at least a portion of the esophageal tube and having a distal opening and a proximal opening, (iii) an endoscopic camera tube extending adjacent the intubation tube, (iii) a balloon extending around a circumference of the esophageal tube and positioned distal of the distal opening of the intubation tube; visualizing the vocal cords of the patient with an endoscopic camera; inflating the balloon to occlude the esophageal opening of the patient; inserting an introducer rod into the intubation tube; advancing the introducer rod through the intubation tube until a distal end of the introducer rod passes through the patient's vocal cords into the patient's trachea; deflating the balloon; and withdrawing the intubation instrument from the patient's mouth while maintaining the introducer rod in place relative to patient's trachea.

In some embodiments, advancing the introducer rod through the intubation tube until a distal end of the introducer rod passes through the patient's vocal cords into the patient's trachea includes: contacting the balloon in an inflated condition with the distal end of the introducer rod.

In some embodiments, the method further comprises: advancing an endotracheal tube along the introducer rod, subsequent to withdrawing the intubation instrument from the patient's mouth, until a distal end of the endotracheal tube passes into the patient's trachea; inflating an endotracheal balloon coupled to the endotracheal tube; and removing the introducer rod from the patient, which includes retracting the introducer rod proximally through the endotracheal tube. The endotracheal balloon and the endotracheal tube are included in an endotracheal system that is separate from the intubation instrument. In some embodiments, the intubation instrument includes only a single balloon.

In some embodiments, the method further comprises inserting an endoscopic camera distally into the endoscopic camera tube; and visualizing, with the endoscopic camera, the distal end of the introducer rod as the introducer rod passes through the patient's vocal cords.

In some embodiments, the method further comprises suctioning contents from the stomach of the patient through the esophageal tube subsequent to inflating the balloon. In some embodiments, the method further comprises coupling an artificial manual breathing unit to the intubation tube and ventilating the patient therewith prior to advancing the introducer rod into the intubation tube. In some embodiments, the method further comprises suctioning contents of the patient through the intubation tube prior advancing the introducer rod into the intubation tube.

In some embodiments, the method further comprises suctioning contents of the patient through at least one of a pair of airway tubes positioned adjacent the intubation tube during the intubation procedure; and passing oxygen in the distal direction through at least one of the pair of airway tubes positioned adjacent the intubation tube during the intubation procedure.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is a perspective view of an intubation instrument having an esophagus-occluding intubation balloon shown in the deflated condition;

FIG. 2 is a perspective view of an introducer rod configured to be inserted in an intubation tube of the intubation instrument of FIG. 1;

FIG. 3 is a perspective view of an esophageal system including an esophageal tube configured to be advanced along the introducer rod of FIG. 2 after the intubation instrument of FIG. 1 has been removed from the patient;

FIG. 4 is a perspective view the intubation instrument of FIG. 1 showing the esophagus-occluding intubation balloon in the inflated condition to prevent the introducer rod of FIG. 2 from enter the esophagus;

FIG. 5 is a cross section view of the intubation instrument take along the line 5-5 of FIG. 4 showing a cover surrounding components of the intubation instrument, the components including the esophageal tube, the intubation tube, the endoscopic camera tube, the airway tubes, and the balloon insufflation tube;

FIG. 6 is a cross section view of the intubation instrument take along the line 6-6 of FIG. 4 showing a cover surrounding the esophageal tube and the airway tube, wherein anterior-posterior opening apertures of an airway tube are shown proximal of the distal opening of an airway tube;

FIG. 7 is a diagrammatic view of a surgical system showing that the surgical system includes the endotracheal system of FIG. 3 and an intubation system, which includes the intubation instrument of FIGS. 1 and 4 and the introducer rod of FIG. 2; and

FIG. 8 is a side cutaway view, wherein the anatomy of the patient is cutaway to show a side view of the intubation system, and wherein the introducer rod is inserted in the intubation tube such that an endoscopic camera at a distal opening of the endoscopic camera tube has a direct line of sight to a distal end of the introducer rod when the introducer rod is positioned in the patient's trachea and the intubation balloon is positioned in the patient's esophagus.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

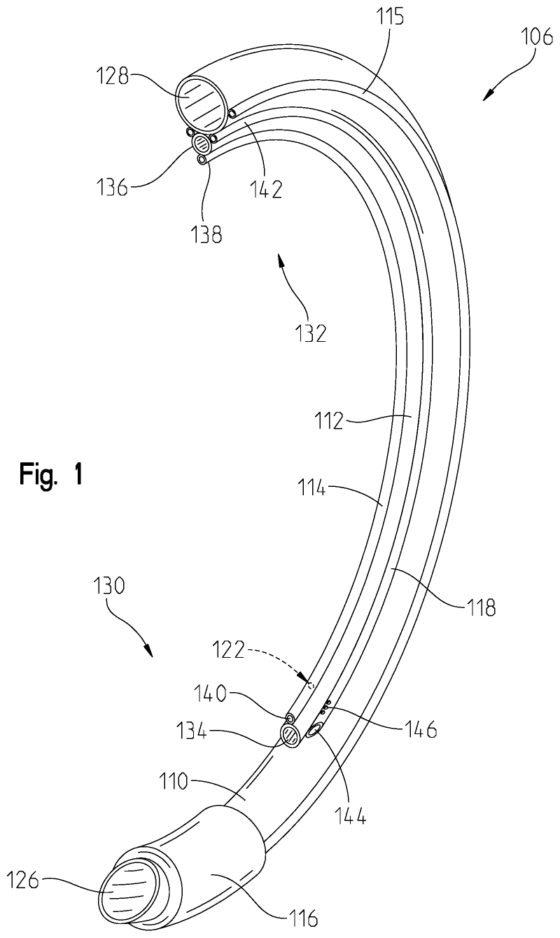

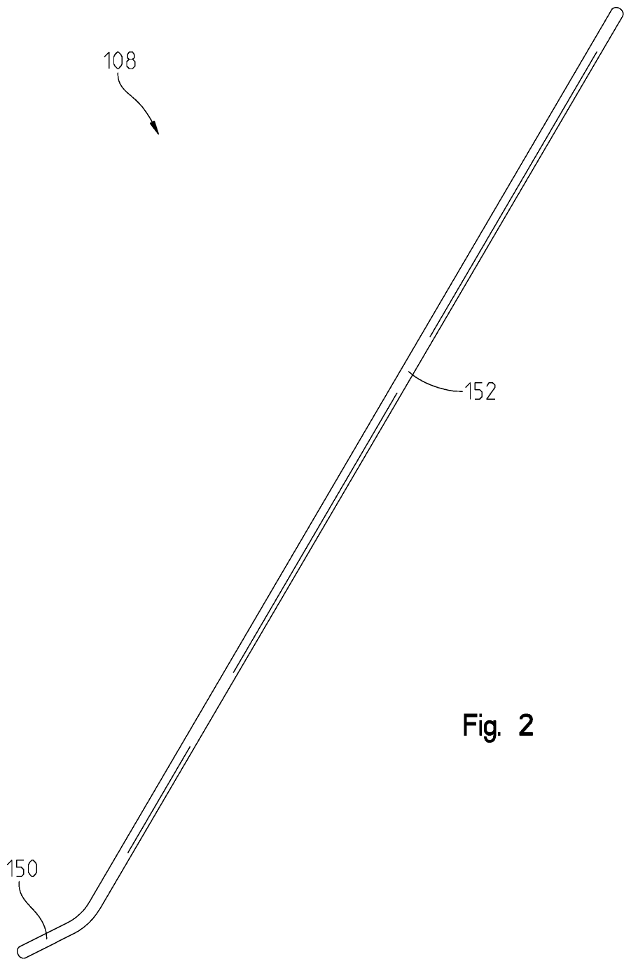

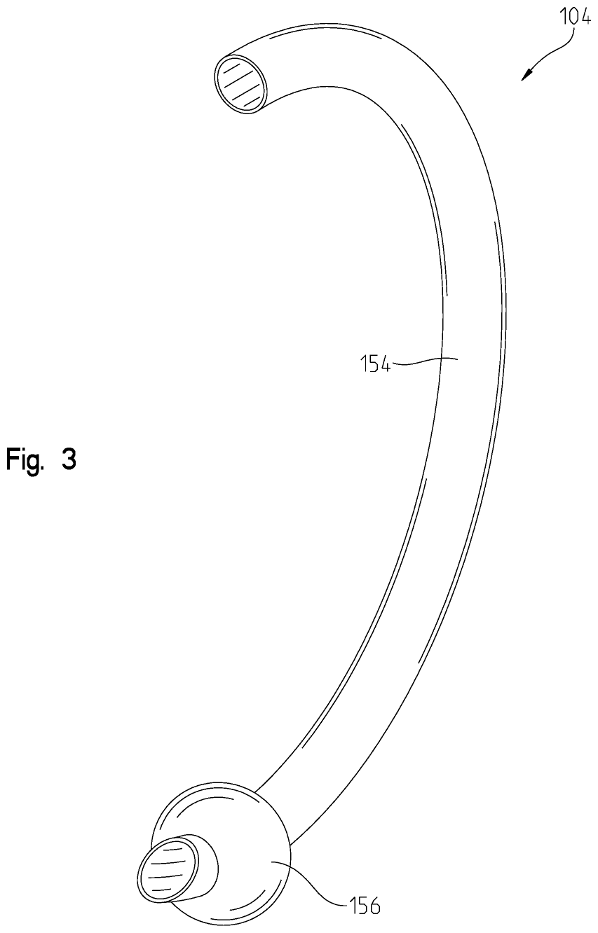

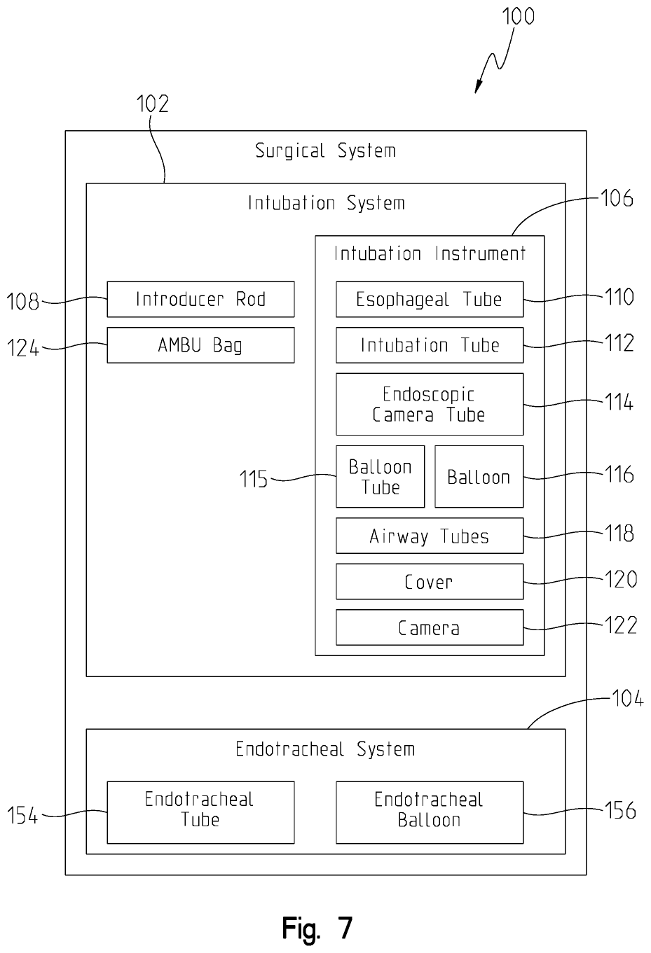

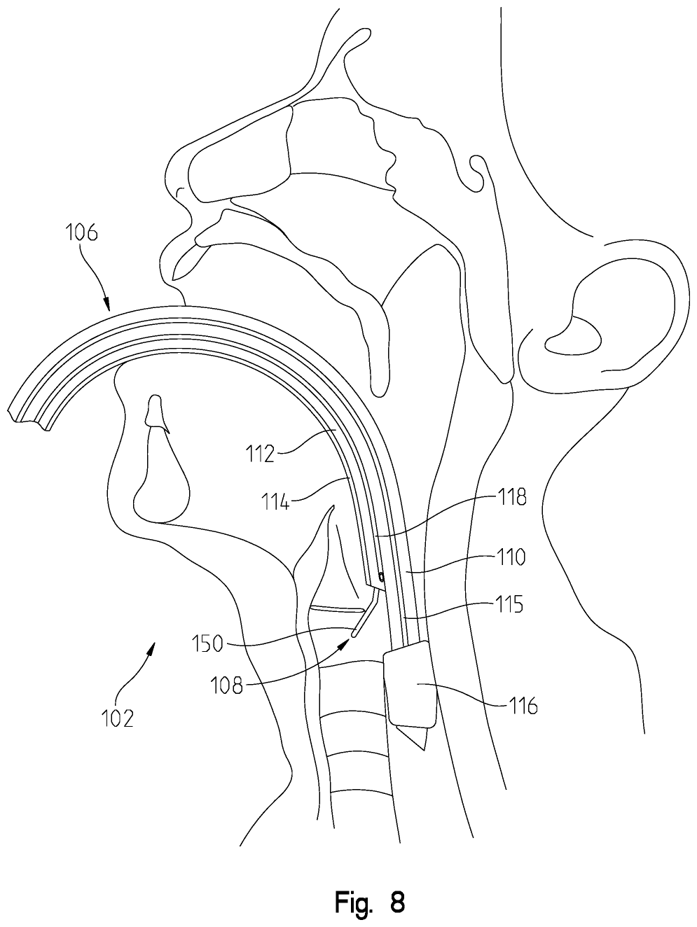

FIG. 1 is an exemplary embodiment of an intubation instrument 106. FIG. 2 is an exemplary embodiment of an introducer rod 108 for use with the intubation instrument 106. The intubation instrument 106 and the introducer rod 108 are included in an intubation system 102. FIG. 3 is an exemplary embodiment of an endotracheal system 104 for use with the introducer rod 108. The intubation system 102 and the endotracheal system 104 are included in a surgical system 100, which may be used to perform procedures including difficult intubations, for example, for a patient with multiple traumas, obesity, or excessive secretions or blood in the oropharynx. The intubation instrument 106 is used to perform the intubation portion of the procedure. It should be appreciated that the intubation instrument 106 and the endotracheal system 104 are each configured to be used with the introducer rod 108; however, the intubation instrument 106 and the endotracheal system 104 are not suitable for use directly with one another during the surgical procedure. This is different than conventional systems, in which various intubation and endotracheal devices are used in simultaneously, thereby providing advantageous over conventional systems as described below. A diagrammatic view of the surgical system 100 is shown in FIG. 7.

Referring again to FIG. 1, the intubation instrument 106 includes a curved esophageal tube 110, an intubation tube 112, an endoscopic camera tube 114, a balloon insufflation tube 115, a balloon 116, and airway tubes 118. The esophageal tube 110 includes a distal opening 126 and a proximal opening 128. The distal direction is indicated by the arrow 130 and the proximal direction is indicated by the arrow 132. In the illustrative embodiment shown in FIG. 1, the esophageal tube 110 has a greater length and a greater diameter than the intubation tube 112. Although in other embodiments, the diameter of the intubation tube 112 may be the same or greater than that of esophageal tube 110. The varied diameters, in particular the smaller diameters relative to other devices such as an endotracheal tube, may be advantageous to prevent any attempt by a user to insert an endotracheal tube or other device improperly into a passageway of the intubation instrument 106. For example, in one embodiment, the length of the esophageal tube 110 is in the range of 24-26 cm, and the esophageal tube 110 has a diameter in the range of 0.5-0.6 cm.

In the illustrative embodiment of FIG. 1, the intubation tube 112 includes a distal opening 134 and a proximal opening 136. The intubation tube 112 extends along a portion of the esophageal tube 110 matching the contour thereof. The intubation tube 112 is positioned anterior to the esophageal tube 110. In the illustrative embodiment, the intubation tube 112 has a length in the range of 18-20 cm and a diameter in the range of 0.6-0.8 cm.

The endoscopic camera tube 114 extends adjacent at least a portion of the intubation tube 112 and is positioned anterior to the intubation tube 112. In illustrative embodiment, the endoscopic camera tube 114 extends along the entire length of the intubation tube 112 such that the two are coterminous, each ending at their respective openings. As shown in FIG. 1, the endoscopic camera tube 114 includes a proximal opening 138 and a distal opening 140, which is adjacent the distal opening 134 of the intubation tube 112. In some embodiments, the endoscopic camera tube 114 has a length in the range of 14-18 cm and a diameter in the range of 0.3-0.4 cm. In some embodiments, the intubation instrument 106 includes an endoscopic camera 122, which may be embodied as a nasopharyngeal scope that is flexible and insertable into the endoscopic camera tube 114; however, this disclosure contemplates any endoscope with suitable flexibility, diameter, and length. When the endoscopic camera 122 is inserted into the endoscopic camera tube 114, the endoscopic camera 122 is positioned at the distal opening 140 of the endoscopic camera tube 114. The position of the distal opening 140 relative to the other components of the intubation instrument 106 is advantageous such that it allows a direct line of sight for the endoscopic camera 122, unobstructed by other components of the intubation instrument 106, to the vocal cords, which are positioned at the superior portion of the trachea.

The balloon 116 may be referred to as an intubation balloon or an esophagus-occluding intubation tube. In any event, the balloon 116 is the only balloon included in the intubation instrument 106. The balloon 116 extends around a circumference of the esophageal tube 110, is fixed thereto, and is positioned distal of the distal opening 134 of the intubation tube 112. In some embodiments, the balloon 116 is positioned between the distal opening 126 of the esophageal tube and the distal opening 134 of the intubation tube. Further, in some embodiments, the a distal edge of balloon 116 is coterminous with the distal opening 126 of the esophageal tube 110. In the illustrative embodiment, the balloon 116 is approximately 2 cm in length and includes a maximum inflation volume in the range of 40-50 ML.

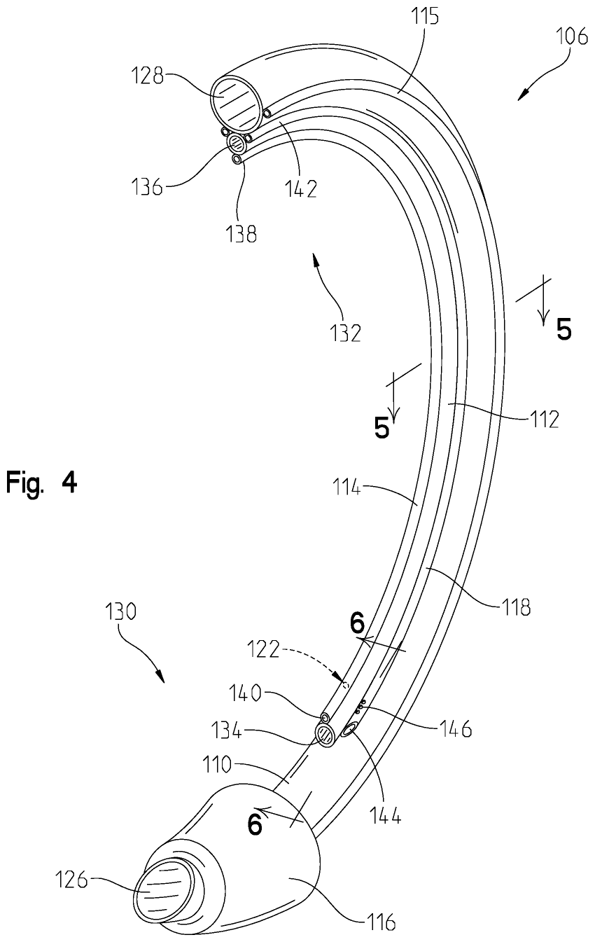

The balloon 116 is movable between a deflated condition (FIG. 1) and an inflated condition (FIG. 4). In use, the intubation instrument 106 is orally inserted through the mouth of a patient such that a portion thereof is located internal to the patient. In this instance, when the balloon 116 is in the inflated condition, the balloon 116 fully occludes the esophageal opening of the patient, and when the balloon 116 is in the deflated condition, the balloon does not fully occlude the esophageal opening of the patient. In some embodiments, when the balloon 116 is in the inflated condition, the intubation instrument 106 is fixed relative to the patient's esophagus, and when the balloon 116 is in the deflated condition, the intubation instrument 106 is movable relative to the patient's esophagus.

As described above, the intubation instrument 106 includes only a single balloon 116, which is advantageous because additional balloons are unnecessary for the surgical procedure performed with the intubation instrument 106 and such additional balloons may cause obstruction or difficulty for a user attempting to properly position, insert, or withdraw the intubation instrument 106.

The balloon insufflation tube 115 is coupled at a distal end to the intubation balloon 116 to facilitate airflow thereto and at a proximal end to a syringe configured to urge air distally through the balloon insufflation tube 115 to the balloon 116. The balloon insufflation tube 115 cooperates with the syringe to move the balloon 116 between the inflated and deflated conditions. In the illustrative embodiment, the balloon insufflation tube 115 extends along the esophageal tube 110; however, in other embodiments, the balloon insufflation tube 115 may be positioned directly adjacent to other components of the intubation instrument 106. In the illustrative embodiment, the balloon insufflation tube 115 is approximately 30 cm in length and approximately 1 cm in diameter.

In the illustrative embodiment, the airway tubes 118 are positioned on opposite sides of the intubation tube 112, and such positioning provides the advantage of allowing suction or positive airflow at the distal opening 140 of the endoscopic camera tube 114 where the endoscopic camera 122 is positioned. This is advantageous because suction or positive airflow at such a location ensures that the endoscopic camera 122 maintains an unobstructed line of sight in the direction of the patient's vocal cords and provides oxygen to the patient effectively. The airway tubes 118 extend along a portion of the esophageal tube 110 and are positioned anterior thereof. In some embodiments, the airway tubes 118 are coterminous with one or both of the esophageal tube 110 and the intubation tube 112. In the illustrative embodiment, the airway tubes 118 each have a length in the range of 18-20 cm and have a diameter of approximately 0.2 cm. Each airway tube 118 is configured to facilitate suction in the proximal direction and configured to urge positive airflow in the distal direction. The airway tubes 118 may be coupled to a wall-mounted (or other) oxygen unit to provide oxygenation (i.e. positive airflow in the distal direction) during the intubation process described herein. In illustrative embodiments, each airway tube includes a proximal opening 142, a distal opening 144, and a plurality of apertures 146 (e.g., three apertures, as shown in FIG. 1). The plurality of apertures 146 are defined in the body of the airway tubes 118 proximal to the distal opening 144. It should be appreciated that while the proximal openings 146 of the airway tubes 118 are both shown in FIG. 1, the remaining portion of the left airway tube 146 is eclipsed by the esophageal tube 110; however, the left airway tube 146 is a mirror image of the right airway tube 146 and otherwise identical thereto.

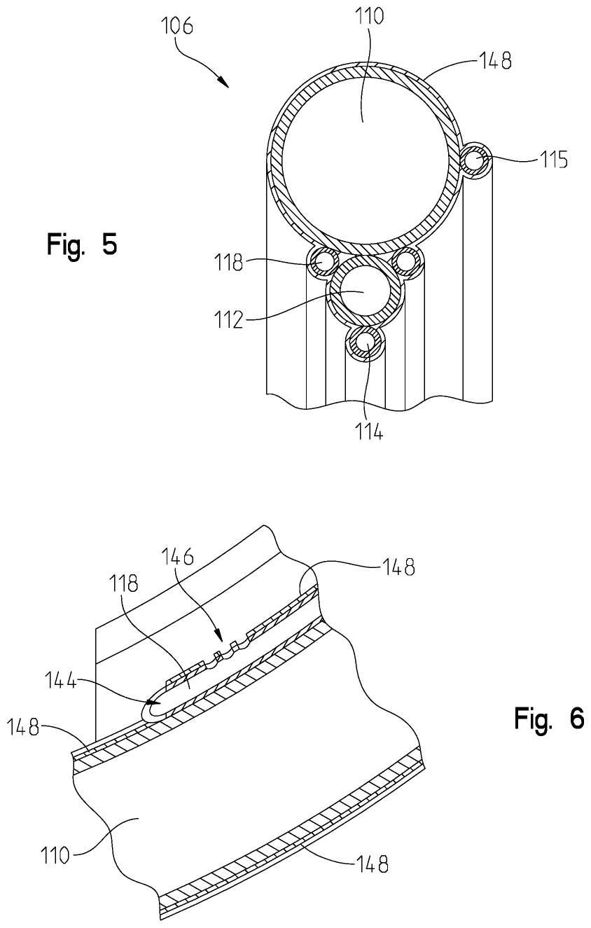

As shown in FIGS. 5 and 6, in some embodiments, the intubation instrument 106 includes a cover 148 surrounding the exposed portions of each of the esophageal tube 110, the intubation tube 112, the endoscopic camera tube 114, the balloon insufflation tube 115, and the pair of airway tubes 118. In particular, FIG. 6 provides a detailed view of the distal opening 144 and the apertures 146 of an airway tube 118 surrounded by the cover 148. In the illustrative embodiment, the cover 148 matches the contours of each component that it surrounds. In this embodiment, the cover 148 may be referred to as a coating at times. In another embodiment, the cover 148 has a cylindrical cross-section with an outer diameter in the range of 1.8-2.0 cm and a length in the range of 24-28 cm. In this embodiment, each of the esophageal tube 110, the intubation tube 112, the endoscopic camera tube 114, the balloon insufflation tube 115, and the pair of airway tubes 118 are surrounded by the cover 148. In various embodiments, the cover 148 may be formed of silicon, plastic, or another other material suitable for surrounding and facilitating use of the other structures and arrangements of the intubation instrument 106 described herein.

Referring now to FIG. 2, the introducer rod 108 is an elongated rod having a length greater than that of the intubation tube 112. In the illustrative embodiment, the introducer rod is a flexible rod having a diameter of approximately 0.2 cm and a length in the range of 34-36 cm. In the illustrative embodiment, a distal end 150 of the introducer rod 108 is angled approximately 10-30 degrees relative to a body portion 152 of the introducer rod, and the body portion 152 is straight. In some embodiments, the introducer rod 108 is straight along its entire length. In some embodiments, the body portion 152 is at least ten times the length of the distal end 150.

It should be appreciated that the introducer rod 108 is insertable in the intubation tube 112 of the intubation instrument 106, and that in such an arrangement, introducer rod 108 and the intubation tube 112 are movable relative to one another. It should also be appreciated that the balloon 116 is arranged relative to the intubation tube 112 such that the introducer rod 108 is configured to contact the balloon 116 during movement of the introduce rod 108 relative to the intubation instrument 106 when the balloon 116 is positioned in the patient's esophagus. It should also be appreciated that when the introducer rod 108 is positioned in the intubation tube 112 and the distal end 150 of the introducer rod 108 is positioned distal of the distal opening 134 of the intubation tube 112, the endoscopic camera 122 has a line of sight to the distal end 150 of the introducer rod 108 when the balloon 116 is in the inflated condition in the patient's esophagus.

As described above, in addition to the intubation system 102, the surgical system 100 also includes an endotracheal system 104. As shown in FIG. 3, the endotracheal system 104 includes an endotracheal tube 154 and an endotracheal balloon 156. When the introducer rod 108 is positioned in the patient's trachea, the endotracheal tube 154 may be advanced along the introducer rod 108 such that the introducer rod 108 guides the endotracheal system 104 into the patient's trachea. The endotracheal tube 154 is sized and shaped such that it cannot pass through any tube of the intubation instrument 112 to be inserted in the patient's trachea. This feature is advantageous over conventional systems because the intubation instrument 112 must be removed before introducing the endotracheal system 104 to the patient, eliminating potential for error.

A method of using the surgical system 100 may include an intubation procedure as described herein. A user may insert the intubation instrument 106 through a patient's mouth, visualize the vocal cords of the patient with the endoscopic camera 122, inflate the balloon 116 such that the balloon 116 occludes the esophageal opening of the patient, insert the introducer rod 108 into the intubation tube 112, advance the introducer rod 108 through the intubation tube 112 until the distal end 150 of the introducer rod 108 passes through the vocal cords of the patient, deflate the balloon 116, and withdraw the intubation instrument 106 from the patient's mouth while maintaining the introducer rod 108 in place relative to the trachea and vocal cords.

As an advantage of aspects of this invention, it should be appreciated that when the balloon 116 is inflated, the introducer rod 108 cannot be advanced into the patient's esophagus. This is especially helpful in difficult intubations, in which the introducer rod 108 may have a tendency to move into the esophagus rather than into the trachea of a patient. To this point, in some embodiments, when a user advances the introducer rod 108 through the intubation tube 112, the distal end 150 of the introducer rod 108 may contact the balloon 116, when the balloon 116 is in the inflated condition, prior to entering the trachea. The angled distal end 150 of the introducer rod 108 may further facilitate movement of the introducer rod 108 into the patient's trachea, especially during contact with the balloon 116.

In some embodiments, the method of using the surgical system 100 includes additional steps subsequent to withdrawing the intubation instrument 106. For example, a user may pass the endotracheal tube 154 over the introducer rod 108 until an endotracheal balloon 156 of the endotracheal system 104 passes into the patient's trachea. The user may inflate the endotracheal balloon 156 coupled to the endotracheal tube 154 in the patient's trachea and subsequently remove the introducer rod 108 from the patient by advancing the introducer rod 108 proximally through the endotracheal tube 154.

In some embodiments, a user may insert the endoscopic camera 122 distally into the endoscopic camera tube 114, prior to visualizing, with the endoscopic camera 122, the distal end 150 of the introducer rod 108 as the introducer rod 108 passes into the patient's trachea.

In difficult intubation procedures, further complications may arise, and the intubation system 102 includes components sufficient to provide a solution to such complications. For example, in some embodiments, a user may couple an artificial manual breathing unit (AMBU or AMBU bag) 124 to the intubation tube 112. Subsequently, the user may ventilate the patient with the AMBU bag prior to inserting the introducer rod 108 into the intubation tube 112. In some embodiments, the user may suction contents from the stomach of the patient through the esophageal tube 110 subsequent to inflating the balloon 116. In some embodiments, the user may suction the contents from the patient through the intubation tube 112 prior advancing the introducer rod 108 into the intubation tube 112. In some embodiments, the user may suction contents of the patient through at least one of a pair of airway tubes 118 positioned adjacent the intubation tube 112 during the intubation procedure. The suction step using the airway tube 118 may occur prior to the visualization step. In some embodiments, the user may couple the airway tubes 118 to a wall mounted (or other) oxygen unit and pass oxygen in the distal direction through at least one of the airway tubes 118 during the intubation procedure.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. An intubation system comprising: an introducer rod and an intubation instrument, each configured to move relative to one another; wherein the intubation instrument includes:
an esophageal tube that is curved and has a distal opening and a proximal opening;
an intubation tube extending adjacent a portion of the esophageal tube and having a distal opening and a proximal opening;
an endoscopic camera tube extending adjacent the intubation tube and including a proximal opening and a distal opening adjacent the distal opening of the intubation tube;
a balloon fixed relative to the esophageal tube and extending around a circumference of the esophageal tube, wherein the balloon is distal of the distal opening of the intubation tube;
wherein the introducer rod is flexible and insertable through the intubation tube;
wherein the balloon is movable between: (a) an inflated condition, in which the balloon fully occludes the esophageal opening of a patient and (b) a deflated condition in which the balloon does not fully occlude the esophageal opening of the patient when positioned in the esophageal opening of the patient.

2. The intubation system of claim 1, wherein the balloon is arranged relative to the intubation tube such that the introducer rod is configured to contact the balloon during movement of the introduce rod relative to the intubation instrument when the balloon is positioned in the esophagus of the patient.

3. The intubation system of claim 1, wherein the intubation instrument includes only a single balloon.

4. The intubation system of claim 1, further comprising an endoscopic camera positioned at the distal opening of the endoscopic camera tube in an arrangement such that the endoscopic camera has a line of sight to the vocal cords of the patient.

5. The intubation system of claim 4, wherein when the introducer rod is positioned in the patient's trachea and a distal end of the introducer rod is positioned distal of the distal opening of the intubation tube, the endoscopic camera has a line of sight to the distal end of the introducer rod when the balloon is in the deflated condition and in the inflated condition.

6. The intubation system of claim 5, further comprising a pair of airway tubes positioned on opposite sides of the intubation tube;
wherein each airway tube is configured to facilitate suction in the proximal direction and urge airflow in the distal direction.

7. The intubation system of claim 6, wherein each airway tube includes a proximal opening, a distal opening, and a plurality of apertures defined proximal of the distal opening.

8. The intubation system of claim 6, further comprising a cover surrounding each of the esophageal tube, the intubation tube, the endoscopic camera tube, a balloon insufflation tube coupled to the balloon, and the pair of airway tubes.

9. The intubation system of claim 8, wherein the cover comprises a silicone material.

10. The intubation system of claim 1, wherein the balloon is positioned between the distal opening of the esophageal tube and the distal opening of the intubation tube.

11. A surgical system, comprising the intubation system of claim 1 and an endotracheal system, the endotracheal system comprising:
an endotracheal tube configured to be advanced along the introducer rod when the introducer rod is positioned in the patient's trachea; and
wherein the endotracheal tube is sized and shaped such that it cannot pass through the intubation tube or the esophageal tube of the intubation instrument to be inserted in the trachea of the patient.

12. A method of performing an procedure on a patient comprising:
inserting an intubation instrument through the mouth of the patient, the intubation instrument including: (i) an esophageal tube that is curved, (ii) an intubation tube extending along at least a portion of the esophageal tube and having a distal opening and a proximal opening, (iii) an endoscopic camera tube extending adjacent the intubation tube, (iii) a balloon extending around a circumference of the esophageal tube and positioned distal of the distal opening of the intubation tube;
visualizing the vocal cords of the patient with an endoscopic camera;
inflating the balloon to occlude the esophageal opening of the patient;
inserting an introducer rod into the intubation tube;
advancing the introducer rod through the intubation tube until a distal end of the introducer rod passes through the patient's vocal cords into the patient's trachea;
deflating the balloon; and
withdrawing the intubation instrument from the patient's mouth while maintaining the introducer rod in place relative to patient's trachea.

13. The method of claim 12, wherein advancing the introducer rod through the intubation tube until a distal end of the introducer rod passes through the patient's vocal cords into the patient's trachea includes:
contacting the balloon in an inflated condition with the distal end of the introducer rod.

14. The method of claim 12, further comprising:
advancing an endotracheal tube along the introducer rod, subsequent to withdrawing the intubation instrument from the patient's mouth, until a distal end of the endotracheal tube passes into the patient's trachea;
inflating an endotracheal balloon coupled to the endotracheal tube; and
removing the introducer rod from the patient, which includes retracting the introducer rod proximally through the endotracheal tube;
wherein the endotracheal balloon and the endotracheal tube are included in an endotracheal system that is separate from the intubation instrument.

15. The method of claim 14, wherein the intubation instrument includes only a single balloon.

16. The method of claim 12, further comprising:
inserting an endoscopic camera distally into the endoscopic camera tube; and
visualizing, with the endoscopic camera, the distal end of the introducer rod as the introducer rod passes through the patient's vocal cords.

17. The method of claim 12, further comprising:
suctioning contents from the stomach of the patient through the esophageal tube subsequent to inflating the balloon.

18. The method of claim 12, further comprising:
coupling an artificial manual breathing unit to the intubation tube and ventilating the patient therewith prior to advancing the introducer rod into the intubation tube.

19. The method of claim 12, further comprising:
suctioning contents of the patient through the intubation tube prior advancing the introducer rod into the intubation tube.

20. The method of claim 12, further comprising:
suctioning contents of the patient through at least one of a pair of airway tubes positioned adjacent the intubation tube during the intubation procedure; and
passing oxygen in the distal direction through at least one of the pair of airway tubes positioned adjacent the intubation tube during the intubation procedure.
